(19) United States
(12) Patent Application Publication
(10) Pub. No.: US 2013/0052719 A1
(43) Pub. Date: Feb. 28, 2013

(54) PHOTOBIOREACTOR FOR MASS CULTURE OF MICROALGAE, AND METHOD FOR CULTURING MICROALGAE BY USING SAME

(71) Applicants: Choul-Gyun Lee, Seoul (KR); Sang-Min Lim, Incheon (KR); Jae-Han Bae, Incheon (KR); Kwang-kuk Cho, Daejeon (KR); Z-Hun Kim, Incheon (KR); Sin-Ae Kang, Incheon (KR); Hye-Jung Kim, Incheon (KR)

(72) Inventors: Choul-Gyun Lee, Seoul (KR); Sang-Min Lim, Incheon (KR); Jae-Han Bae, Incheon (KR); Kwang-kuk Cho, Daejeon (KR); Z-Hun Kim, Incheon (KR); Sin-Ae Kang, Incheon (KR); Hye-Jung Kim, Incheon (KR)

(73) Assignee: INHA-INDUSTRY PARTNERSHIP INSTITUTE, Incheon (KR)

(21) Appl. No.: 13/580,635

(22) PCT Filed: Feb. 22, 2011
(86) PCT No.: PCT/KR11/01147

§ 371 Date: Nov. 12, 2012

(30) Foreign Application Priority Data
Feb. 22, 2010 (KR) 10-2010-0015584
Aug. 9, 2010 (KR) 10-2010-0076416
Aug. 9, 2010 (KR) 10-2010-0076433

(51) Int. Cl. C12M 1/42; C12N 1/12

(52) U.S. Cl. 435/257.1; 435/292.1

(57) ABSTRACT

The present invention relates to a photobioreactor for mass culture of microalgae. The present invention provides a photobioreactor for mass culture of microalgae, comprising: a culture container comprising external walls which comprise an optical filter zone capable of selective penetration or blocking of a part of wavelength or region from sunlight and/or a heat conversion zone for selectively absorbing a part of wavelength or region from sunlight to convert the same into heat, and a reaction chamber which is three-dimensionally formed so as to accommodate microalgae and is an inner space restricted by the external walls; and a coupling means for connecting a floating means which is connected to the culture container or the culture container to the bottom of the water or a structure on the water, thereby locating the culture container near the surface of the water for the exposure of the culture container to sunlight.

### DESCRIPTION

1. Technical Field

The present invention relates to a photobioreactor for mass culturing of microalgae and a method for culturing microalgae by using the same, and more particularly, relates to a photobioreactor for mass culturing of microalgae having a filtering function of transmitting or blocking a light of a specific wavelength or region.

2. Background Art

Microalgae are a photosynthetic unicellular microorganism and may produce various kinds of organic materials such as proteins, carbohydrates, lipids, etc. Particularly, microalgae is evaluated as an optimal organism for making a high value product such as functional polysaccharides, carotenoids, vitamins, unsaturated fatty acids, etc, and for removing carbon dioxide, which is the main factor of a global warming. The microalgae has a shorter doubling time than upland plants and so the amount of the carbon dioxide that is the main culprit of the global warming may be effectively reduced, grows up rapidly in poor surroundings, and directly uses a combustion gas from a power plant or a factory.

Concerning the removal of the carbon dioxide, microalgae producing biological energy for replacing limited energy sources, such as fossile fuels, has received much attention because microalgae has been known to fix the carbon dioxide so as to accumulate within the body as lipids. Lots of researches on producing biodiesel using the accumulated lipids have been conducted. In order to remove the carbon dioxide or to accomplish a mass production of useful products such as bio energy by using the microalgae, culturing the microalgae is required to be conducted on a large scale at a high concentration. Accordingly, techniques concerning establishment on a large-sized culturing equipment is essentially required.

Commonly, a large-sized outdoor pond is set up or a pipe-type photobioreactor is installed as plant facilities for culturing the microalgae. In addition, various types of the photobioreactors set up indoor instead of outdoor have been used as culturing equipments for culturing the microalgae.

### DISCLOSURE OF THE INVENTION

### Technical Problem

However, the photobioreactor set up outdoor (open type photobioreactor) has some defects concerning the contamination by other microorganisms, an excessive cost for establishing, maintaining, repairing and operating, and the lack of an applicable land space. Accordingly, an object of an economic production of bio energy using a product induced from the microalgae may not be accomplished.

In order to achieve a commercially available mass culturing, the ensuring economic feasibility is the first consideration. Therefore, a development on a culturing technique for culturing the microalgae at a high concentration and for easily enlarging the size at a low cost is acutely required.

According to the present invention for solving various problems including the above-described problems, a photobioreactor for culturing microalgae having an extremely high efficiency of light utilization, and a method of culturing the microalgae are provided. The objects are only for illustration and should not be used to limit the scope of the present invention.

### Technical Solution

In accordance with an aspect of the present invention, a photobioreactor for mass culturing of microalgae, comprising a culturing vessel comprising an outer wall including a light filtering region for selectively transmitting or blocking a portion of wavelength or region of sunlight and/or a heat transforming region for transforming selectively absorbing a portion of the wavelength into heat, and a reaction chamber as a three dimensional inner space defined by the outer wall for accommodating the microalgae; and

a floating unit for positioning the culturing vessel at just below a surface of water or at an appropriate depth in the water, or a combining unit for connecting the culturing vessel with a surface under the water, an underwater structure, or a structure on the water, in order to expose the culturing vessel to an appropriate intensity of the sunlight for growing the microalgae and in order to protect from waves is provided.

In accordance with another aspect of the present invention, a photobioreactor for mass culturing of microalgae, comprising a culturing vessel comprising an outer wall including a light filtering region for selectively transmitting or blocking a portion of wavelength or region of sunlight and/or a heat transforming region for selectively absorbing a portion of the wavelength or the region of the sunlight to transform into heat, and a reaction chamber as a three dimensional inner space defined by the outer wall for accommodating the microalgae, the culturing vessel including a first layer and a second layer constituting the outer wall at a different position from the first layer and having a different property from the first layer; and

a floating unit for positioning the culturing vessel near a surface of water, or a combining unit for connecting the culturing vessel with a surface under the water, an underwater structure, or a structure on the water, in order to expose the culturing vessel to an appropriate intensity of the sunlight for growing the microalgae and in order to protect from waves is provided.

In accordance with further another aspect of the present invention, a method of culturing microalgae using a photobioreactor comprising:
(a) injecting culture medium in a culturingvessel of one of the above described photobioreactors, and inoculating microalgae;(b) sealing the culturing vessel, fixing the culturing vessel to a floating unit, a surface under the water, an underwater structure or a structure on the water, and then putting the culturing vessel into seawater or fresh water; and
(c) irradiating light to the microalgae for photosynthesisis provided.

### BRIEF DESCRIPTION OF THE DRAWINGS

FIGS. 1 to 4 are schematic diagrams of photobioreactors in accordance with a first embodiment of the present invention.

FIGS. 5 to 8 are schematic diagrams of photobioreactors in accordance with a second embodiment of the present invention.

FIGS. 9 to 11 are schematic diagrams of photobioreactors in accordance with a third embodiment of the present invention.

FIG. 12 is a schematic diagram of a photobioreactor in accordance with a fourth embodiment of the present invention.

FIG. 13 is a schematic diagram of a photobioreactor in accordance with a fifth embodiment of the present invention.

FIG. 14 is a schematic diagram of a photobioreactor in accordance with a sixth embodiment of the present invention.

FIGS. 15 to 17 are schematic diagrams of photobioreactors in accordance with a seventh embodiment of the present invention.

FIG. 18 is a flow chart for controlling transmission sunlight energy to a photobioreactor by using a light blocking pattern layer of a tape type in accordance with time.

FIGS. 19A and 19B are schematic diagrams on a plate type culturing vessel of a photobioreactor in accordance with an eighth, ninth or eleventh embodiment of the present invention.

FIGS. 20A to 20C are schematic diagrams for illustrating light blocking patterns, which may be formed at one side of a culturing vessel of the photobioreactor in accordance with the eighth embodiment of the present invention.

FIG. 21 is a graph illustrating absorbance of microalgae pigments according to light wavelengths.

FIG. 22 is a schematic diagram illustrating a culturing vessel of a photobioreactor in accordance with a tenth embodiment of the present invention.

FIGS. 23 and 24 are exemplary diagrams on culturing vessels of a photobioreactor in accordance with an embodiment of the present invention.

FIG. 25 illustrates a group of plate type photobioreactors in FIG. 23.

FIG. 26 illustrates a group of cylindrical type photobioreactors in FIG. 24.

### BEST MODE FOR CARRYING OUT THE INVENTION

In accordance with an aspect of the present invention, a photobioreactor for mass culturing of microalgae, including a culturing vessel comprising an outer wall including a light filtering region for selectively transmitting or blocking a portion of wavelength or region of sunlight and/or a heat transforming region for transforming selectively absorbing a portion of the wavelength into heat, and a reaction chamber as a three dimensional inner space defined by the outer wall for accommodating the microalgae; and

a floating unit for positioning the culturing vessel at just below a surface of water or at an appropriate depth in the water, or a combining unit for connecting the culturing vessel with a surface under the water, an underwater structure, or a structure on the water, in order to expose the culturing vessel to an intensity of the sunlight appropriate for growing the microalgae and in order to protect from waves is provided.

In this case, a whole or a portion of the outer wall may include a transparent material. The transparent material may be one of glass, polyvinyl chloride (PVC), polyethyleneterephthalate (PET), acryl, polystyrene (PS), high density polyethylene (HDPE), low density polyethylene (LDPE), polypropylene (PP), polycarbonate (PC), polyamide (PA), or a laminate structure of two or more of them.

The whole or the portion of the outer wall may include a semi-permeable material. The semi-permeable material may be at least one polymer selected from the group consisting of cellulose acetate, cellulose triacetate, cellulose acetate-cellulose triacetate blends, nitrocellulose, gelatin, polyamine, polyimide, poly(ether imide), aromatic polyamide, polybenzimidazole, polybenzimidazolone, polyacrylonitrile, polyacrylonitrile-poly(vinyl chloride) copolymer, polysulfone, polyethersulfone, poly(dimethylphenylene oxide), poly(vinylidene fluoride), polyelectrolyte complexes, polyolefin, poly(methyl methacrylate), polyvinyl alcohol, and a copolymer thereof.

An appropriate depth in the water means a penetrating depth of the sunlight and may be from 1 cm to 50 m, or from 10 cm to 25 m, or from 10 cm to 10 m. When the depth is too large, photosynthesis may not be sufficiently performed and when the depth is too small, the photobioreactor may be broken by strong wind such as typhoon, etc.

In accordance with another aspect of the present invention, a photobioreactor for mass culturing of microalgae, including a culturing vessel comprising an outer wall including a light filtering region for selectively transmitting or blocking a portion of wavelength or region of sunlight and/or a heat transforming region for transforming selectively absorbing a portion of the wavelength into heat, and a reaction chamber as a three dimensional inner space defined by the outer wall for accommodating the microalgae, the outer wall including a first layer and a second layer apart from the first layer and having a different property from the first layer; and

a floating unit for positioning the culturing vessel at just below a surface of water or at an appropriate position in the water, or a combining unit for connecting the culturing vessel with a surface under the water, an underwater structure, or a structure on the water, in order to expose the culturing vessel to an amount of the sunlight appropriate for growing the microalgae and in order to protect from waves is provided.

In this case, the outer wall may include the first layer and the second layer apart from the first layer and having a different property from the first layer. Light transmission properties of the first layer and the second layer may be different from each other. In this case, the wavelength regions of the penetrating light through the first layer and the second layer may be different from each other. Selectively, the first layer and the second layer may have different material penetrating properties from each other. In this case, the first layer may include a semi-permeable layer having a selective permeability with respect to oxygen or carbon dioxide, and the second layer may include a semi-permeable layer having a selective permeability with respect to water and nutrient salts. Selectively, the first layer and the second layer may have different light reflecting properties from each other and the light wavelength penetrated through the first layer may be reflected by the second layer to be supplied into the reaction chamber.

In accordance with another aspect of the present invention, the light filtering region or the heat transforming region may be a pattern having a predetermined shape. In this case, the pattern may be designed to change the shape or density thereof in accordance with the input of a light energy. Selectively, the light filtering region or the heat transforming region may include a light transparent layer and a light blocking pattern layer or a heat transforming pattern layer attached on one side of the outer wall. In this case, the light blocking layer or the heat transforming pattern layer may be a tape possibly attached to or detached from the light transparent layer.

In accordance with further another aspect of the present invention, the light filtering region may be obtained by painting, coating or mixing a coloring agent. In this case, the light filtering region may be obtained by painting, coating or mixing one pigment selected from the group consisting of lead chromate (PbCrO₄), yellow iron oxide (FeO(OH) or Fe₂O₃H₂O), cadmium yellow (CdS or CdS+ZnS), titanium yellow (TiO₂NiOSb₂O₃), chrome orange (PbCrO₄PbO), molybdenum orange (PbCrO₄PbMoO₄PbSO₄), red iron oxide (Fe₂O₃), red lead oxide (Pb₃O₄), cadmium red (CdS+CdSe), manganese violet (NH₄MnP₂O₇), Prussian blue (Fe(NH₄)Fe(CN)₆xH₂O), ultramarine blue (Na_{6˜8}Al₆Si₆O₂₄S_{2˜4}), cobalt blue (CoOAl₂O₃), chrome green (lead chromate+Prussian blue), emerald green (Cu(CH₃CO₂)₂Cu(AsO₂)₂), and a mixture of two or more of them. Selectively, the pigment may include natural pigments excluding heavy metals. The natural pigments may include one of gardenia yellow, polygonum indigo blue, Broussonetia kazinokii blue, carthamus red, Schisandra chinesis red, gallnut yellow, mugwort green, astaxanthin, anthocyanin, picoerythrin, xanthophyl, fucoxanthin, phycocyanin resveratrol, carotenoid, benzoquinone, shikonin, alazanine, anthraquinone, naphtoquinone, flavin, isoflavin and a mixture of them.

Meanwhile, in accordance with another aspect of the present invention, the heat transforming region may include a light heat transforming material. The light heat transforming material may have a function of transforming a light energy into heat energy (see Korean Patent Publication No. 2004-0071142). The light heat transforming material may absorb infrared rays, near infrared rays, visible rays or ultraviolet rays. The light heat transforming material may be an organic or inorganic pigment or dye, an organic coloring agent, a metal, a metal oxide, a metal carbonate or a metal borate.

In accordance with another aspect of the present invention, a portion of or a whole of the culturing vessel may be additionally coated with water repellent to prevent the generation of humidity or a water drop.

In accordance with another aspect of the present invention, a plurality of the light filtering region may be arranged horizontally or vertically one by one, or in a lattice pattern so as to transmit two or more wavelength regions within the sunlight region so as to possibly control a wavelength ratio.

In accordance with further another aspect of the present invention, the structure on the water may include an aquafarm equipment, a buoy, a light buoy, an underwater plant, a floating wave absorbing revetment, a barge, or a mega-float. The underwater plant may include a wind power generation equipment, a tide power generation equipment, a wave power generation equipment, a marine heliport or an oil prospecting ship. The underwater structure may include various cables, a gas pipe or an oil pipeline.

In exemplary embodiments, a shape keeping frame may be additionally disposed at an inside or outside of the culturing vessel to keep the three dimensional shape of the culturing vessel.

In accordance with still further another aspect of the present invention, the culturing vessel and the floating unit are connected by using at least one length adjustable rope, and a weighing pendulum may cling to a bottom portion of the culturing vessel. The pendulum weight and the rope length may be controlled to control the depth of the culturing vessel into the water. Selectively, a gas supplying pipe connected to the bottom portion of the culturing vessel and a gas supplying unit for supplying external air through the gas supplying pipe to the culturing vessel may be additionally included. The microalgae may be uniformly dispersed within the culturing vessel through a mixing reaction of bubbles. Selectively, the culturing vessel may have an inverse conical shape and the gas supplying pipe and the weighing pendulum may be connected to an apex thereof.

Meanwhile, the culturing vessel may include an upper vertical cylindrical portion and a lower inverse conical portion to promote the mixing of cells through a supplied gas and to prevent precipitation of the cells. The gas supplying pipe and the weighing pendulum may be connected to an apex of the inverse conical portion. In accordance with further another aspect of the present invention, at least two of the culturing vessels may be arranged above and below and the weighing pendulum may be cling to at least one of the culturing vessel including a lowermost culturing vessel.

In accordance with further another aspect of the present invention, the floating unit may include at least one pair of connecting points, and the culturing vessel may have a length larger than a width. The connecting points where both end portions in lengthwise direction make a pair are connected by at least one rope so that the culturing vessel may be overturned freely by waves or movements of seawater. Selectively, the culturing vessel is formed to have an elongated cylindrical shape and is connected to the floating unit, the underwater structure, the structure on the water or the surface under the water while lying along the surface of the water in parallel. In accordance with another embodiment, the culturing vessel may be formed to have a middle cylindrical portion and a pair of conical portions at both ends of the middle cylindrical portion. The rope may be connected to each of the apexes of the conical portions.

In accordance with further another aspect of the present invention, a method of culturing microalgae comprises

(a) injecting culture medium in a culturing vessel of one of photobioreactors as above described, and inoculating microalgae;

(b) sealing the culturing vessel, fixing the culturing vessel to a floating unit and then putting the culturing vessel into seawater or fresh water; and

(c) irradiating light to the microalgae for photosynthesis.

The microalgae may include chlorella, Haematococcus, Botryococcus, Scenedesmus, Nannochloropsis, Nannochloris, spirulina, Chlamydomonas, Phaeodactylum, Dunaliella, Schizochytrium, Nitzschia, Tetraselmis, Porphyridium, cyanobacteria. The microalgae may produce in the photobioreactor, carotenoids, mycobionts, phycobili-proteins, lipids, carbohydrates, unsaturated fatty acids, proteins, etc.

### MODE FOR CARRYING OUT THE INVENTION

Hereinafter, preferred embodiments of the present invention will be explained in more detail referring to attached drawings. The following embodiments and drawings are illustrated for embodying the present invention and it should be understood that the content of the present invention is not limited to the following embodiments and drawings.

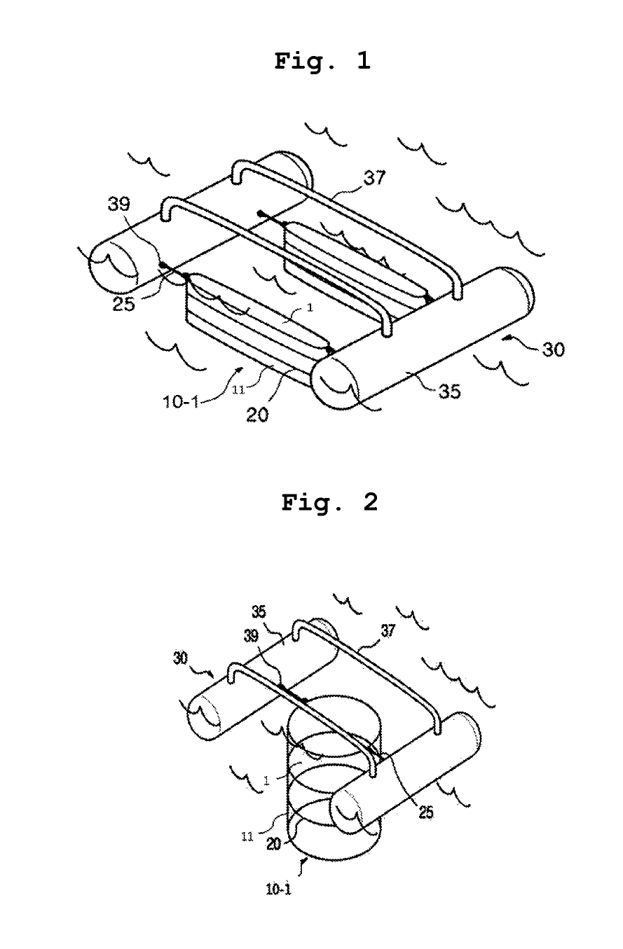

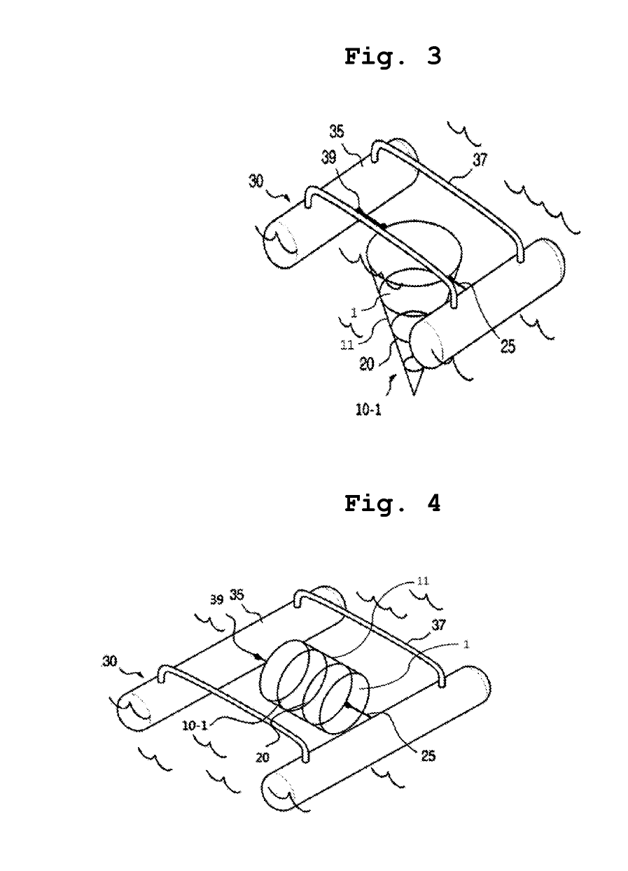

FIGS. 1 to 4 are schematic diagrams of photobioreactors in accordance with the first embodiment of the present invention. The photobioreactor in accordance with the first embodiment of the present invention may include a culturing vessel 10-1 and a floating unit 30 connected to the culturing vessel 10-1 for positioning the culturing vessel 10-1 near the surface of water in order to expose the culturing vessel 10-1 to a sunlight.

Particularly, the culturing vessel 10-1 is formed as a layer having a designated thickness and has a three-dimensional structure including an outer wall 11 including a light filtering region for selectively transmitting or blocking a portion of wavelength or region of sunlight or a heat transforming region for transforming selectively absorbing a portion of the wavelength into heat, and a reaction chamber 1 defined by the outer wall 11 for accommodating the microalgae as a culturing space. When the culturing vessel 10-1 is formed by using a material such as a flexible glass, plastic and semi-permeable layer, a shape keeping frame 20 for keeping the three-dimensional structure of the culturing vessel 10-1 may be additionally included in or out of the culturing vessel 10-1 to keep the three dimensional culturing space stably. Particularly, when the culturing vessel 10-1 is formed by using the semi-permealbe layer possibly filtering a designated wavelength region, discharged excreta from the microalgae while growing and metabolite inhibiting the growth of the microalgae may dissolve in the seawater and may be eliminated along with discharging seawater. Accordingly, a separate purifying work to remove the excreta and the metabolite inhibiting the growth of the microalgae may not be necessary and so, the change of a culture medium may not be required. In addition, the microalgae may also use carbon dioxide dissolved in the seawater for photosynthesis. Oxygen produced after the photosynthesis may be exhausted into the air through the semi-permeable layer. The shape of the culturing vessel 10-1 may be changed according to the properties of the microalgae and the size of the culturing without restriction.

According to the major characteristic of the present invention, the culturing vessel 10-1 may be formed by using various materials such as the glass, the plastic, the semi-permeable layer for passing the seawater while blocking the microalgae, etc. and the total or a portion of the culturing vessel 10-1 includes the light filtering region for transmitting or blocking a designated wavelength or region and/or a heat transforming region for transforming selectively absorbing a light having a designated wavelength into heat. The light filtering region or the heat transforming region may be provided with the whole outer wall 11 or may constitute a portion of the outer wall 11. The light filtering region or the heat transforming region may be formed by coating or painting the outer wall 11 or by mixing a pigment while forming the material of the outer wall 11. Thus the culturing vessel 10-1 may supply or block the microalgae with a designated wavelength region and the production of the mycobionts of the microalgae may be improved and the metabolite may be increased according to the wavelength. With respect to the wavelength or region of the light to be penetrated or blocked, the wavelength or region of red, blue, green etc. may be penetrated or blocked depending on the efficiency of a light energy of the light wavelength by the microalgae. The heat transforming region may include a light heat transforming material to transform the light not used for the photosynthesis into the heat to improve the culturing efficiency and the photosynthesis of the microalgae. Particularly, the heat transforming region may decrease the lowering degree of the temperature of a culture medium. In this case, the culturing of the microalgae may be conducted in winter time. Meanwhile, water repellent coating of the upper portion of the culturing vessel may be additionally performed to prevent the forming of a water drop due to the evaporation of the water.

The floating unit 30 is provided to dispose the culturing vessel 10-1 near the surface of the water so that the culturing vessel 10-1 may be exposed to the sunlight. As illustrated in FIG. 1, the floating unit 30 may include a pair of floating members 35 apart from each other with a designated distance and a connecting frame 37 for connecting the pair of the floating members 35 while keeping the designated distance. Here, various modification of the floating unit 30 may be included according to the shape and size of the culturing vessel 10-1 without restriction.

Selectively, the culturing vessel may be connected through a combining unit (not illustrated) to a structure on the water, an underwater structure or a surface under the water (not illustrated). The combining unit may be formed using various elements including a rope, an iron chain, a steel wire, etc. The structure on the water may include an aquafarm equipment, a buoy, a light buoy, an underwater plant, a floating wave absorbing revetment, a barge, or a mega-float. The underwater plant may include a wind power generation equipment, a tide power generation equipment, a wave power generation equipment, a marine heliport or an oil prospecting ship. The underwater structure may include a cable, a gas pipe or an oil pipeline.

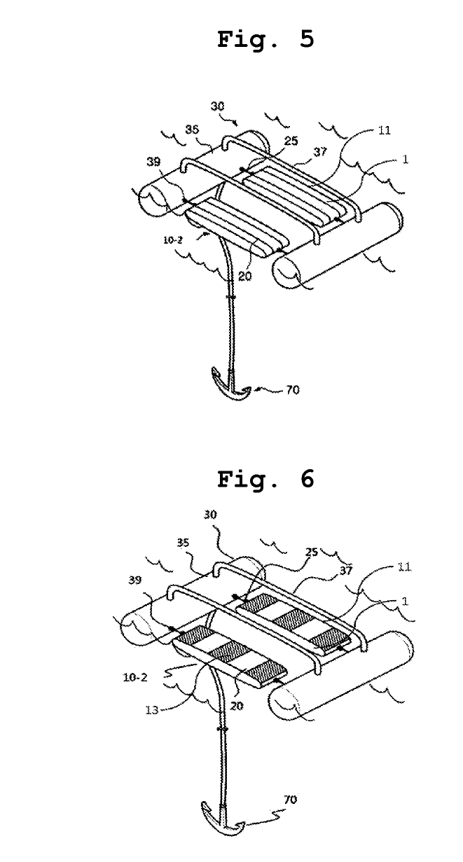

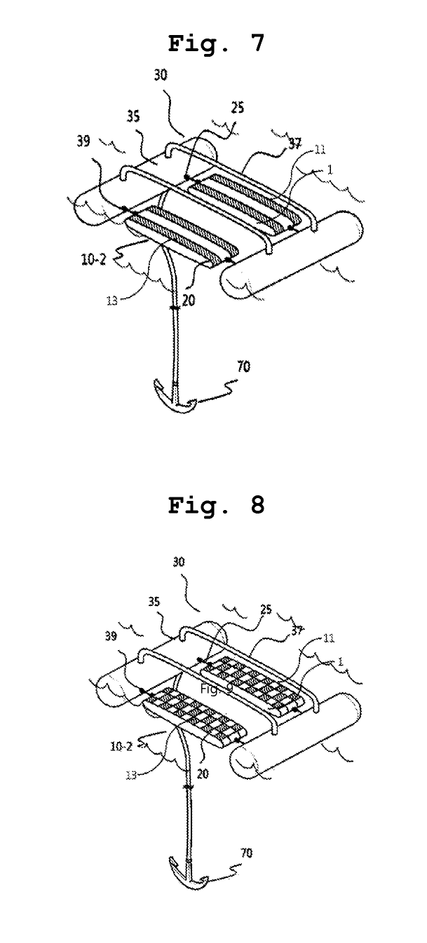

FIGS. 5 to 8 are schematic diagrams of photobioreactors in accordance with a second embodiment of the present invention. The photobioreactor in accordance with the second embodiment of the present invention may include a culturing vessel 10-2 and a floating unit 30 as in the first embodiment and the culturing vessel 10-2 and the floating unit 30 may be connected by using at least one length adjustable rope 25 as illustrated in FIG. 2

The culturing vessel 10-2 may be manufactured so as to transmit or block only one wavelength region from the sunlight or may be manufactured so as to transmit two or more wavelength regions from the sunlight. Light filtering regions 13 of the culturing vessel 10-2 may be alternately arranged in a horizontal or vertical direction, may be formed by mixing portions transmitting designated wavelength regions, or may be arranged in a lattice shape to control the ratio of the designated wavelength region applied to the microalgae.

Selectively, the total or a portion of the light filtering region 13 may be replaced with a heat transforming region 13 for absorbing a designated wavelength region from the sunlight and then transforming into heat. In order to prevent the forming of the water drop, a water repellent coating may be conducted with respect to a portion or the total of the upper portion of the culturing vessel.

When the culturing vessel 10-2 is formed by using a material such as a flexible glass, plastic and semi-permeable layer, a shape keeping frame for keeping the three-dimensional structure of the culturing vessel 10-2 may be additionally included in or out of the culturing vessel 10-2 to keep the three dimensional culturing space.

In addition, the photobioreactor in accordance with the second embodiment may additionally include a fixing unit 70 fixed to the surface under the water and connected to a floating unit 30 to limit a moving range of the photobioreactor. The fixing unit 70 may prevent the movement of the photobioreactor depending on the movement of the seawater and the fresh water so as not to deviate from a manageable restricting area. The fixing unit 70 may be formed by using a material having a high density and may have a shape similar to an anchor so as to be fixed to the surface under the water easily. However, the shape of the fixing unit 70 is not restricted.

Selectively, the culturing vessel may be connected through a combining unit (not illustrated) to a structure on the water, an underwater structure or the surface under the water (not illustrated) instead of the floating unit 30 in accordance with another embodiment of the present invention. The combining unit may include various elements such as a rope, an iron chain, a steel wire, etc. The structure on the water may include an aquafarm equipment, a buoy, a light buoy, an underwater plant, a floating wave absorbing revetment, a barge, or a mega-float. The underwater plant may include a wind power generation equipment, a tide power generation equipment, a wave power generation equipment, a marine heliport or an oil prospecting ship. The underwater structure may include a cable, a gas pipe or an oil pipeline.

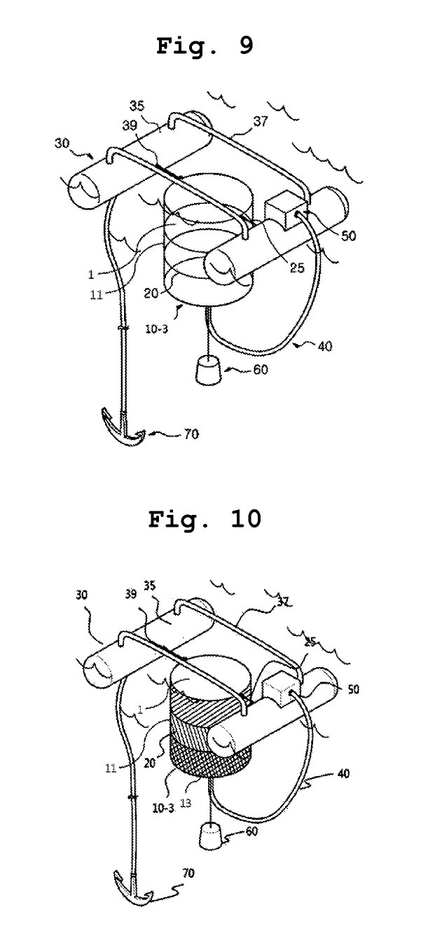

FIGS. 9 to 11 are schematic diagrams of photobioreactors in accordance with a third embodiment of the present invention. As illustrated in FIG. 9, the photobioreactor in accordance with the third embodiment may include a cylindrical culturing vessel 10-3 and a floating unit 30. In addition, the culturing vessel 10-3 and the floating unit 30 may be connected by at least one of a length adjustable rope 25 as illustrated in the second embodiment and may include a weighing pendulum 60 cling to the bottom portion of the culturing vessel 10-3.

The photobioreactor in accordance with the third embodiment may additionally include a gas supplying pipe 40 connected to the bottom portion of the culturing vessel 10-3 and a gas supplying unit 50 for supplying an external air to the culturing vessel 10-3 through the gas supplying pipe 40. Accordingly, the microalgae may be uniformly dispersed within the culturing vessel 10-3 through a mixing function by bubbles in the culturing chamber of the culturing vessel 10-3 and the culturing environment of the microalgae may be kept satisfactorily. Practically, the mixing function of the culturing vessel 10-3 may be naturally obtained by the movement of the seawater because of sea currents, a tidal range, etc. Therefore, the above-described gas supplying may be applied when a favorable mixing function is required.

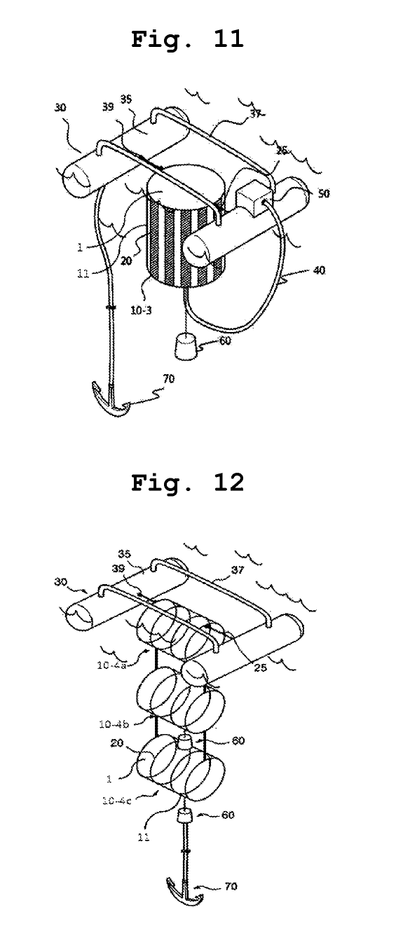

The culturing vessel 10-3 may be formed to transmit or block only a designated wavelength region from the sunlight. In addition, as illustrated in FIGS. 10 and 11, in order to transmit two or more wavelength regions within the sunlight region, a plurality of the light filtering region 13 of the culturing vessel 10-3 may be alternately arranged in a horizontal direction or a vertical direction, or the filtering region 13 may be crossly arranged in a lattice shape to control the ratio of specific wavelength regions applied to the microalgae. Selectively, the total or a portion of the light filtering region 13 may be replaced with a heat transforming region 13 for absorbing the specific wavelength region from the sunlight and then transforming into heat. Meanwhile, in order to prevent the forming of water drops, water repellent coating may be conducted at the total or a portion of the upper portion of the culturing vessel.

When the culturing vessel 10-3 is formed by using a material such as a flexible glass, plastic and semi-permeable layer, a shape keeping frame 20 for keeping the three-dimensional structure of the culturing vessel 10-3 may be additionally included in or out of the culturing vessel 10-3 to confirm the three dimensional culturing space.

Further, in the photobioreactor in accordance with the third embodiment, both of the gas supplying pipe 40 and the weighing pendulum 60 may be connected to the apex of the cylindrical culturing vessel 10-3. Different from the second embodiment, the fixing unit 70 is fixed to the surface under the water, while being connected to the apex of the culturing vessel 10-3 to limit the moving range of the photobioreactor.

Selectively, in accordance with another embodiment of the present invention, the culturing vessel may be connected through a combining unit (not illustrated) to a structure on the water, an underwater structure or the surface under the water (not illustrated) instead of the floating unit 30 in accordance with another embodiment of the present invention. The combining unit may include various elements such as a rope, an iron chain, a steel wire, etc. The structure on the water may include an aquafarm equipment, a buoy, a light buoy, an underwater plant, a floating wave absorbing revetment, a barge, or a mega-float. The underwater plant may include a wind power generation equipment, a tide power generation equipment, a wave power generation equipment, a marine heliport or an oil prospecting ship. The underwater structure may include a cable, a gas pipe or an oil pipeline.

FIG. 12 is a schematic diagram of a photobioreactor in accordance with a fourth embodiment of the present invention. The photobioreactor in accordance with the fourth embodiment of the present invention includes three culturing vessels 10-4 side by side and above and below, which have cylindrical shape and are arranged in parallel with the surface of the water as illustrated in FIG. 12. The number of the culturing vessels 10-4 is not limited. However, the depth of the culturing vessel 10-4 may be restricted appropriately considering the arrival of the sunlight necessary for the microalgae conducting the photosynthesis.

Particularly, the photobioreactor may include at least one weighing pendulum 60 cling to at least one culturing vessel 10-4 including the lowermost culturing vessel 10-4c among the culturing vessels 10-4. Referring to FIG. 12, the weighing pendulums 60 cling to the lowermost culturing vessel 10-4c and the middle culturing vessel 10-4b are illustrated. However, the photobioreactor may include the weighing pendulum 60 clung only to the lowermost culturing vessel 10-4, or may include the weighing pendulums 60 clung to all of the culturing vessels 10-4a, 10-4b and 10-4c. That is, the weighing pendulum 60 may be essentially clung to the lowermost culturing vessel 10-4c to keep the overall arrangement and the balance. However, the weighing pendulum may be selectively clung to the remaining culturing vessels 10-4a and 10-4b. Referring to FIG. 12, only three culturing vessels are illustrated. However, the number of the culturing vessels may increase. Meanwhile, in order to prevent the forming of water drops, water repellent coating may be conducted at the total or a portion of the upper portion of the culturing vessel.

Selectively, in accordance with another embodiment of the present invention, the culturing vessel may be connected through a combining unit (not illustrated) to a structure on the water, an underwater structure or the surface under the water (not illustrated) instead of the floating unit 30 in accordance with another embodiment of the present invention. The combining unit may include various elements such as a rope, an iron chain, a steel wire, etc. The structure on the water may include an aquafarm equipment, a buoy, a light buoy, an underwater plant, a floating wave absorbing revetment, a barge, or a mega-float. The underwater plant may include a wind power generation equipment, a tide power generation equipment, a wave power generation equipment, a marine heliport or an oil prospecting ship. The underwater structure may include a cable, a gas pipe or an oil pipeline.

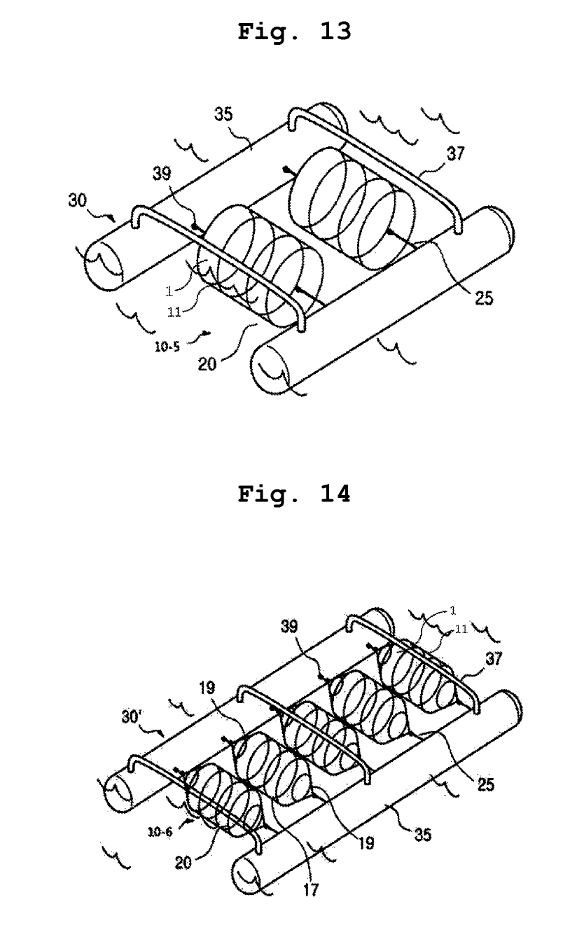

FIG. 13 is a schematic diagram of a photobioreactor in accordance with a fifth embodiment of the present invention. The photobioreactor in accordance with the fifth embodiment of the present invention, as illustrated in FIG. 13, includes elongated cylindrical culturing vessels 10-5 provided in parallel with the surface of the water and connected to a floating unit 30. In this case, the culturing vessel 10-5 may be easily overturned by the wave or the movement of the seawater. Meanwhile, in order to prevent the forming of water drops, water repellent coating may be conducted at the total or a portion of the upper portion of the culturing vessel.

Selectively, in accordance with another embodiment of the present invention, the culturing vessel may be connected through a combining unit (not illustrated) to a structure on the water, an underwater structure or the surface under the water (not illustrated) instead of the floating unit 30 in accordance with another embodiment of the present invention. The combining unit may include various elements such as a rope, an iron chain, a steel wire, etc. The structure on the water may include an aquafarm equipment, a buoy, a light buoy, an underwater plant, a floating wave absorbing revetment, a barge, or a mega-float. The underwater plant may include a wind power generation equipment, a tide power generation equipment, a wave power generation equipment, a marine heliport or an oil prospecting ship. The underwater structure may include a cable, a gas pipe or an oil pipeline.

FIG. 14 is a schematic diagram of a photobioreactor in accordance with a sixth embodiment of the present invention. The photobioreactor in accordance with the sixth embodiment of the present invention, as illustrated in FIG. 14, includes culturing vessels 10-6 having a middle cylindrical portion 17 and a pair of conical portions 19 at both end portions of the cylindrical portion, and connected to a floating unit 30′ through a rope 25 connected to an apex of the conical portion 19. When comparing with the culturing vessel 10-5 in accordance with the fifth embodiment, the culturing vessel 10-6 may be overturned even more easily by the wave or the movement of the seawater. Meanwhile, in order to prevent the forming of water drops, water repellent coating may be conducted at the total or a portion of the upper portion of the culturing vessel.

The photobioreactor in accordance with the sixth embodiment, as illustrated in FIG. 14, includes enlarged floating unit 30′ lengthwise and a large numbers of connecting ring pairs 39 provided at the floating unit 30′. The connecting ring pairs 39 are connected with the culturing vessels 10-6, respectively and the size of the floating unit may be easily enlarged. The width of the floating unit 30′ may also be enlarged even though not illustrated. Accordingly, the size enlargement for the commercial and mass culturing may be expected.

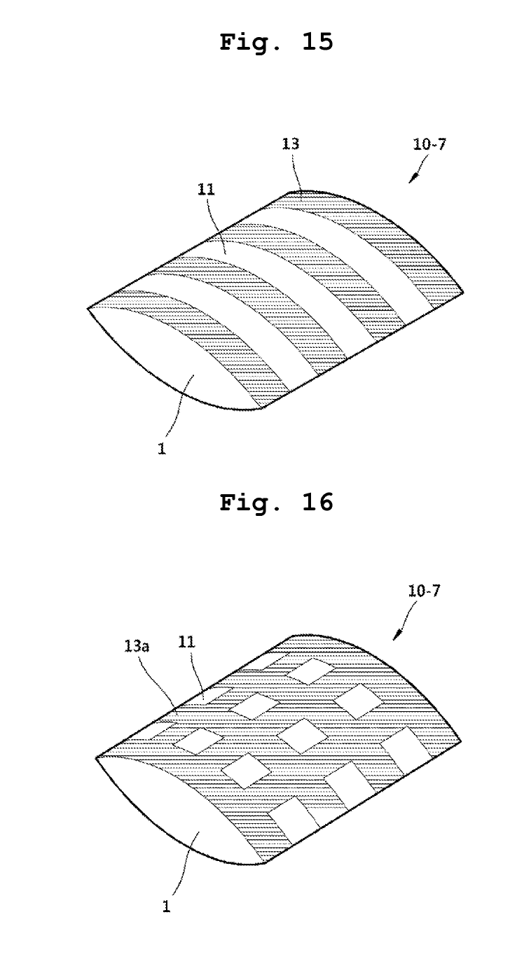

FIGS. 15 to 17 are schematic diagrams of photobioreactors in accordance with a seventh embodiment of the present invention.

In FIG. 15, a culturing vessel 10-7 having a cylindrical shape is illustrated. Referring to FIG. 15, the culturing vessel 10-7 in accordance with exemplary embodiments includes an outer wall 11 formed by a layer having a designated thickness and a reaction chamber 1 as an inner space defined by the outer wall 11 for accommodating microalgae. In this case, one side of the outer wall may include a light filtering region 13 for selectively transmitting or blocking a wavelength region from supplied light to the culturing vessel 10-7. As illustrated in FIGS. 15 and 16, the light filtering region 13 may be formed as a light filtering pattern 13a having a specific shape. Meanwhile, in order to prevent the forming of water drops, water repellent coating may be conducted at the total or a portion of the upper portion of the culturing vessel.

The total or a portion of the outer wall of the culturing vessel 10-7 may include a semi-permeable layer. Oxygen, nitrogen, carbon dioxide, etc. in the air may penetrate the semi-permeable layer and the water, nutrient salts, etc. in the seawater may penetrate the semi-permeable layer. However, the penetration of the microalgae may be blocked.

Accordingly, when the microalgae accommodated in the culturing vessel formed by using the semi-permeable layer is floating on the seawater, the microalgae may be naturally supplied with necessary materials from external environment, while being isolated from the external environment.

Meanwhile, the outer wall 11 may basically transmit light. Accordingly, when the culturing vessel is exposed to a light source, for example, the sun, the sunlight from the sun may penetrate the outer wall 11 and may be supplied to the microalgae accommodated in the reaction chamber 1.

In this case, the outer wall 11 may include a light filtering pattern 13a possibly reducing a portion of the light energy supplied from the light source. The photobioreactor in accordance with the seventh embodiment of the present invention may control the supplied light energy by using the shape or the density of the light filtering pattern 13a formed at the outer wall 11. For example, when the supplied light energy is high, the size of the pattern of the light filtering pattern 13a may be increased or the density of the light filtering pattern 13a may be increased to decrease the light energy passing through the light filtering pattern 13a. When the supplied light energy is low, the contrary method to this may be applied. Different ranges of the light energy may be necessary according to the kind of the microalgae. Even for one kind of the microalgae, different ranges of the light energy may be necessary according to the step during conducting the culturing. Particularly, an inducing production process from Haematococcus to astaxanthin may proceed in two steps. In the first step, light energy of a relatively low intensity may be supplied to sufficiently produce mycobionts and in the second step, light energy of a high intensity may be supplied to induce the astaxanthin.

In the photobioreactor in accordance with the seventh embodiment of the present invention, an appropriate light filtering pattern 13a exposed to the light source, on the outer wall 11 may be selected to accomplish an optimal light energy condition for culturing the microalgae.

Particularly, a light filtering pattern having a high light blocking ratio may be used at the time or season of high sunlight energy to reduce the sunlight energy supplied to the reaction chamber appropriately, to prevent a light inhibiting phenomenon and to supply an appropriate amount of the light energy to the cells.

Meantime, a light filtering pattern having a low light blocking ratio may be used at the time or season of relatively low sunlight energy to supply an appropriate amount of the sunlight for growing the microalgae to efficiently culture the microalgae.

Particularly, when the photobioreactor in accordance with the seventh embodiment of the present invention is floating on the seawater and is exposed to the sunlight, the amount or the energy of the sunlight supplied to the reaction chamber 1 may be optionally controlled depending on the amount of the solar radiation.

The light filtering pattern 13a may have a shape in which stripes are arranged in parallel with a distance as illustrated in FIG. 15 or may have a lattice arrangement as illustrated in FIG. 16. In this case, the light energy blocked by the light filtering pattern 13a may be controlled by appropriately controlling the width or number of the stripes.

The light filtering patterns in FIGS. 15 and 16 are illustrated as exemplary embodiments and any shape of the light filtering patterns accomplishing the object thereof may be used. Various types including a circular type, a polygonal type, a spiral type, a zigzag type, etc. may be possible.

In exemplary embodiments, a region for absorbing a light wavelength from a light source and transforming to heat (will be called as a heat transforming region, hereinafter) may be formed at a portion or the total of the outer wall of the photobioreactor.

When the culturing vessel 10-7 of the above-described constitution is floating on the seawater or fresh water, the heat transforming region may absorb a part of the light wavelength from a light source during the time or season of low temperature and then transforming to heat to increase an internal temperature of the culturing vessel 10-7 to improve the efficiency of the photosynthesis. Accordingly, the decrease of the efficiency of the photosynthesis of the microalgae due to the decrease of the temperature may be prevented to some degree.

The heat transforming region may be formed on one total surface of the outer wall as in FIG. 17 or may be formed in various patterns as for the light filtering pattern.

When the heat transforming region is formed as a heat transforming pattern, the light energy absorbed by the culturing vessel may be controlled by using the shape or the density of the heat transforming pattern 13a formed at the outer wall 11. Particularly, the pattern shape of the heat transforming pattern 13a may be enlarged or the density thereof may be increased to increase the light energy transformed into the heat by the heat transforming region 13.

The heat transforming pattern may be used in combination with the light filtering pattern. Particularly, a portion of the outer wall may be applied as the light filtering region and the remaining portion may be applied as the heat transforming pattern.

The transparent layer of the outer wall corresponding to the light filtering pattern and/or the heat transforming pattern may be formed by using a material having the light filtering property or the heat transforming property. The material having the light filtering property may be at least one pigment selected from the group consisting of lead chromate (PbCrO₄), yellow iron oxide (FeO(OH) or Fe₂O₃H₂O), cadmium yellow (CdS or CdS+ZnS), titanium yellow (TiO₂NiOSb₂O₃), chrome orange (PbCrO₄PbO), molybdenum orange (PbCrO₄PbMoO₄PbSO₄), red iron oxide (Fe₂O₃), red lead oxide (Pb₃O₄), cadmium red (CdS+CdSe), manganese violet (NH₄MnP₄O₇), Prussian blue (Fe(NH₄)Fe(CN)₆xH₂O), ultramarine blue (Na_{6˜8}Al₆Si₆O₂₄S_{2˜4}), cobalt blue (CoOAl₂O₃), chrome green (lead chromate+Prussian blue), emerald green (Cu(CH₃CO₂)₂Cu(AsO₄)₄), and a mixture of them. The material may be painted or coated on the outer wall or may be mixed with a raw material for forming the outer wall. Selectively, the pigment may include a natural pigment excluding heavy metals and may include gardenia yellow, polygonum indigo blue, Broussonetia kazinokii blue, carthamus red, Schisandra chinesis red, gallnut yellow, mugwort green, astaxanthin, anthocyanin, picoerythrin, xanthophyl, fucoxanthin, phycocyanin resveratrol, carotenoid, benzoquinone, shikonin, alazanine, anthraquinone, naphtoquinone, flavin, isoflavin and a mixture of two or more of them. Meanwhile, the light heat transforming material may absorb infrared rays, near infrared rays, visible rays or ultraviolet rays and may be an organic or inorganic pigment or dye, an organic coloring agent, a metal, a metal oxide, a metal carbonate or a metal borate. Also, the heat transforming material may include a black pigment such as carbon black, a pigment having an absorbing region from the visible rays to the near infrared rays such as phthalocyanine, naphthalocyanine, etc., an organic dye (cyanine dye such as indolenine dye, anthraquinone dye, azulene coloring agent, phthalocyanine dye) and an organic metal compound coloring agent such as a dithiol nickel complex.

Selectively, the light filtering region or the heat transforming region may be obtained by separately attaching a light filtering pattern layer or a heat transforming pattern layer onto the whole or a portion of the outer wall of the culturing vessel. In this case, the light filtering pattern layer or the heat transforming pattern layer may be a tape possibly attached to and detached from the outer wall 11. Meanwhile, the sun is the most important light source for the photobioreactor floating on the seawater or the fresh water for culturing the microalgae. For an efficient culturing of the microalgae, the method of producing the microalgae may be required to be conducted considering the light intensity according to the position of the sun. The season changes according to the revolution of the earth and the degree of the sunlight energy reaching to the surface of the earth may change. Day and night come according to the rotation of the earth and during the day time, the degree of the sunlight energy may change according to the rotation period of the earth.

When an excessive light energy over a designated intensity is supplied to the microalgae using the light energy from the sun, the photosynthetic apparatus may be broken and the photosynthesis may not be conducted any more.

Otherwise, secondary metabolite may be accumulated to overcome the light energy and undesirable product other than the mycobiont may be obtained from the culturing of the microalgae.

Therefore, the intensity of the supplied light energy is required to be controlled appropriately considering the properties of the microalgae.

The properties of the transformation of the absorbed sunlight into heat may change according to the season or the time zone. The heat transforming ratio in winter time when the exposing time to the sun is short and an average temperature is low, is required to be increased when comparing with summer time when the exposing time to the sun is long and the average temperature is high.

In exemplary embodiments, the shape or the density of the light filtering pattern or the heat transforming pattern may be designed and applied considering an average amount of sunshine computed with respect to time in order to settle the above-described problems.

Particularly, when the light filtering layer or the heat transforming layer is the tape, attachment and detachment of the tape is easy. By using this property, the intensity of the supplied light energy from the sun to the photobioreactor or the heat transforming ratio may be controlled.

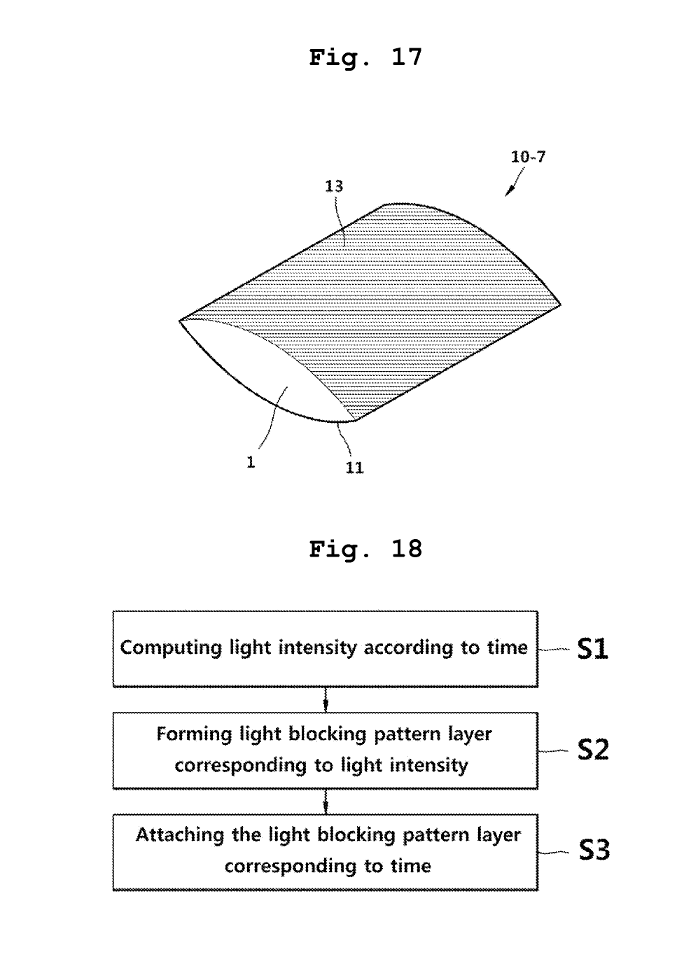

FIG. 18 is a flow chart for controlling applied sunlight energy into a photobioreactor by using a light blocking pattern layer, i.e. a light filtering pattern layer of a tape type in accordance with time.

Referring to FIG. 18, the total time of floating the photobioreactor to expose to the sunlight is divided by a designated period. Then, an average amount of sunshine for each time zone, that is, the light energy is computed (Step S1).

The shape and the density of the light blocking pattern corresponding to the computed light energy is designed and a light blocking pattern layer is formed (Step S2). A plurality of the light blocking pattern layers for each time zone may be obtained. For example, when the amount of the sunshine is excessively high, the shape or the density of the light blocking pattern layer is increased to decrease the light energy passing through the light blocking pattern layer. On the contrary, when the amount of the sunshine is low, the shape or the density of the light blocking pattern layer is decreased to increase the light energy. The light blocking pattern layer formed considering the time zone is attached on one side of the photobioreactor receiving the sunlight (Step S3).

The light filtering pattern layer prepared according to the time zone may be possibly attached and detached. Accordingly, a filtering pattern layer attached to the photobioreactor for a time being may be detached at the beginning of the next time zone and a new light filtering pattern layer corresponding to the new time zone may be attached.

In exemplary embodiments, the supplied light energy to the photobioreactor may be appropriately controlled according to the time zone by using the light filtering pattern and an appropriate amount of the light energy may be supplied to the microalgae. Particularly, for the floating photobioreactor for culturing the microalgae on the sea, the culturing efficiency may be increased and an economic mass culturing of the microalgae may be performed by using the light blocking pattern formed by considering the amount of sunshine according to the time zone.

The above-described method may be applied for the heat transforming pattern layer of a tape type in the same manner.

The culturing vessel of the photobioreactor in accordance with exemplary embodiments may include an outer wall and a reaction chamber having an inner space for accommodating microalgae and a culture medium and defined by the outer wall. The outer wall may include a plurality of layers having different optical properties. Particularly, the outer wall may include a plurality of layers having different light transmitting ratios (or light blocking ratios), different light transmitting properties according to the wavelength region (light filtering property), and different reflectivity. Further, the outer wall may additionally include a plurality of layers having different transmitting ratios according to materials.

FIGS. 19A and 19B are schematic diagrams on a plate type culturing vessel 10-8 of a photobioreactor in accordance with an eighth, ninth or eleventh embodiment of the present invention. Referring to FIGS. 19A and 19B, the plate type culturing vessel 10-8 has a rectangular parallelepiped shape. The outer wall 11 may include different layers having different optical properties and be separately disposed above and below with a distance. In accordance with the eighth embodiment, a first layer 2 having a relatively lower light transmittance may form an upper layer of the rectangular parallelepiped and a second layer 3 having a relatively higher light transmittance may form a lower layer. In this case, the inner space defined by the outer wall including the first layer 1 and the second layer 3 may be a reaction chamber 1 for accommodating microalgae 7 and a culture medium.

When the culturing vessel 10-8 is floating on the seawater or the fresh water as illustrated in FIG. 19, the lower layer may contact the surface of the water or may be submerged to a designated depth. The upper layer may contact the atmosphere and be primarily exposed to a light source.

In FIGS. 19A and 19B, the first layer 2 corresponds to the upper layer and the second layer 3 corresponds to the lower layer. However, when the culturing vessel 10-8 is overturned, the first layer 1 may become the lower layer and the second layer 3 may become the upper layer without restriction. Meanwhile, in order to prevent the forming of water drops, water repellent coating may be conducted at the total or a portion of the upper portion of the culturing vessel.

The culturing vessel 10-8 may additionally include a gas inlet 4 for supplying gas into the reaction chamber 1 and a gas outlet 5 for exhausting gas.

In addition, a sampling port 6 for assembling a sample for confirming the culturing degree of the microalgae may be additionally formed.

The culturing vessel 10-8 of the photobioreactor according to the eighth embodiment of the present invention may control the light energy supplied to the reaction chamber 1 by the time zone and the steps by using the first layer 2 and the second layer 3 having different transmittances. Particularly, when the culturing vessel 10-8 of the photobioreactor in accordance with the eighth embodiment of the present invention is floating on the seawater or the fresh water to expose to the sunlight, the amount or the intensity of the sunlight supplied to the reaction chamber 1 may be controlled according to the amount of the sunshine.

To achieve an effective culturing of the microalgae, the light intensity according to the position of the sun is required to be considered and effectively applied in producing the microalgae. The season changes according to the revolution of the earth and the degree of the sunlight energy reaching to the surface of the earth changes. Day and night come according to the rotation of the earth and during the day time, the degree of the sunlight energy may change according to the rotation period of the earth.

When an excessive light energy over a designated intensity is supplied to the microalgae using the light energy from the sun, the photosynthetic apparatus may be broken and the photosynthesis may not be conducted any more. Otherwise, secondary metabolite may be accumulated to overcome the light energy and undesirable product other than the mycobionts may be obtained from the culturing of the microalgae.

Different ranges of the light energy may be necessary according to the kind of the microalgae. Even for one kind of the microalgae, different ranges of the light energy may be necessary according to the step during conducting the culturing. Particularly, an inducing production process from Haematococcus to astaxanthin may proceed in two steps. In the first step, a relatively low energy may be supplied to sufficiently produce mycobionts and in the second step, a light energy of a high intensity may be supplied to induce the astaxanthin.

In the photobioreactor 10-8 in accordance with the eighth embodiment of the present invention, an appropriate exposing surface to the sunlight during floating on the sea or lake may be selected to accomplish an optimal light energy condition for culturing the microalgae.

Particularly, the first layer 2 having the low transmittance may be provided at the upper portion so as to be exposed to the sunlight during the time zone or the season of the high intensity of the sunlight. In this case, the supplied sunlight energy to the reaction chamber 1 may be decreased to prevent the light lowering phenomenon and to supply appropriate energy to the cells.

Meanwhile, during the time zone or the season of the relatively low intensity of the sunlight, the culturing vessel 10-8 may be overturned to change the positions of the upper layer and the lower layer to expose the second layer 3 having a high transmittance to the sunlight. In this case, an efficient culturing of the microalgae may be conducted through supplying appropriate light energy for the growth of the microalgae.

In exemplary embodiments, each layer of the plurality of the layers having different light transmittance may be formed by using different materials having different light transmittance from each other. Particularly, onto a light transmitting layer, a material having a different light transmittance such as a light blocking film or tape may be attached.

In exemplary embodiments, various types of light filtering patterns for adjusting the light transmittance may be formed on the light transmitting layer to form a plurality of layers having different light transmittance.

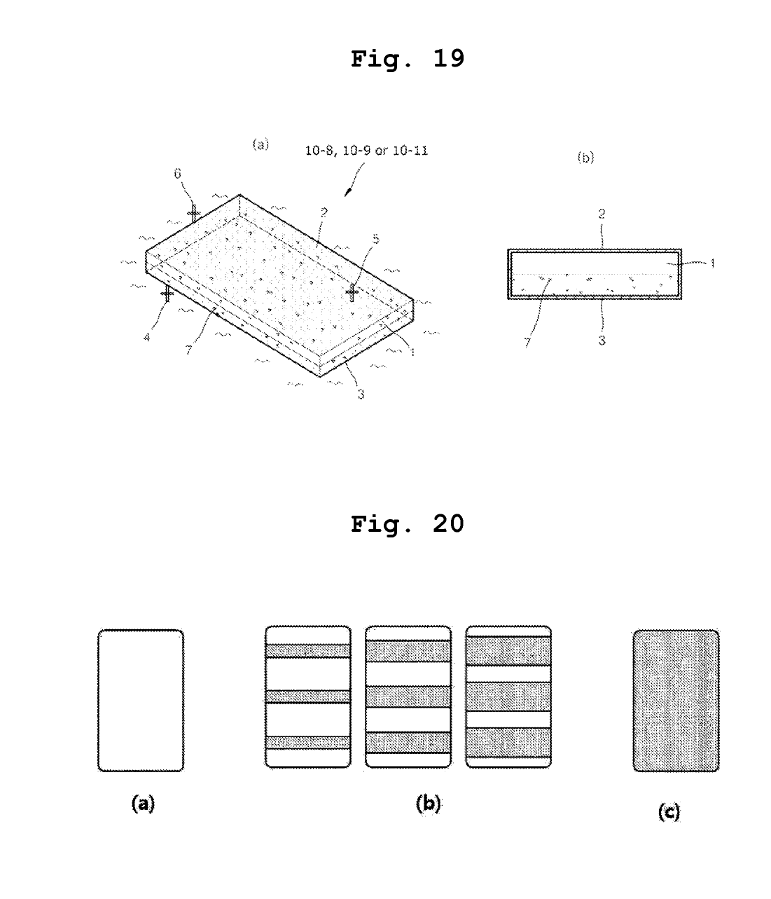

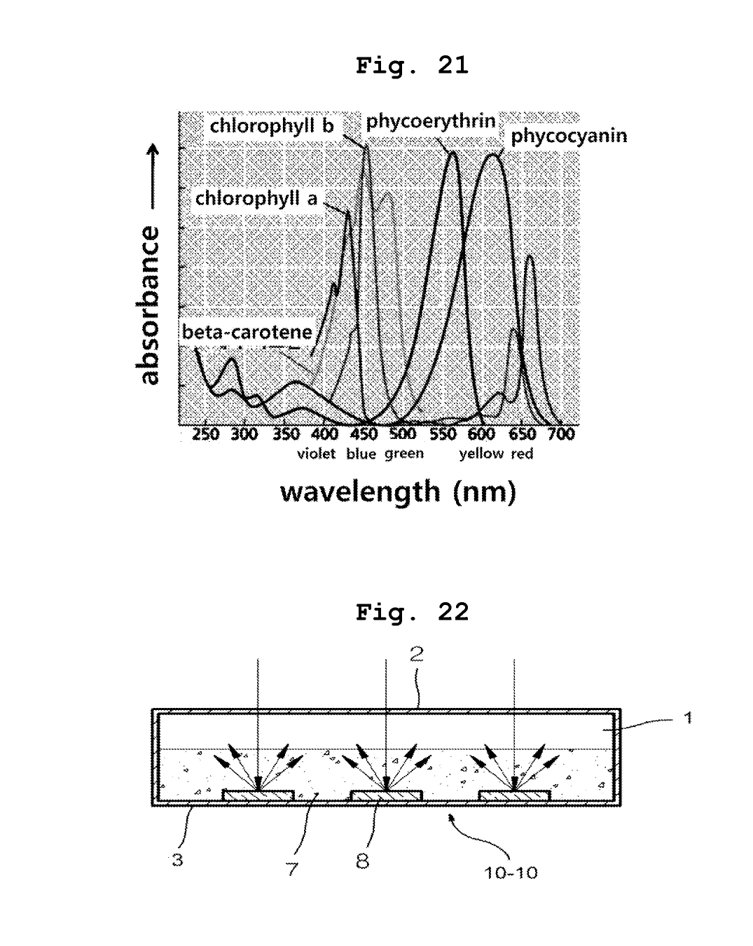

FIGS. 20A to 20C are schematic diagrams for illustrating light filtering patterns (dark portions), which may be formed at one side of a culturing vessel 10-8 of the photobioreactor to adjust the light transmittance.

FIG. 20A illustrates a culturing vessel excluding the light filtering pattern, FIG. 20C illustrates a light filtering pattern for completely blocking the sunlight, and FIG. 20B illustrates a light filtering pattern for blocking the sunlight to a designated ratio, for example, 30%, 50%, or 70%.

The light filtering pattern may have a property of blocking the whole light or of blocking a portion of the light.

In addition, the light filtering pattern may be formed by using a material having a light filtering property or may be formed by attaching a light blocking film or a tape onto the light transmitting layer.

Exemplary embodiments were explained in the preceding referring to the sunlight, however, the present invention is not limited to the sunlight. Light sources such as an LED lamp may be obviously used. The same applies to the following embodiments.

Meanwhile, the light wavelength is one of major factors in culturing the microalgae. The concentration of the mycobiont and the concentration of the metabolites produced from the microalgae may be improved by supplying the light wavelength of a specific wavelength region to the microalgae.

The microalgae may include chlorophyll and various colorings for conducting the photosynthesis. Generally, visible rays between 300 nm to 700 nm wavelength are used for the photosynthesis. Green algae mostly absorbs light wavelength in a red region or a blue region to use in the photosynthesis.

Particularly, one of the green algae, Chlorella illustrates higher growing property when a light wavelength energy in the red region by using a red light emitting diode (680 nm) when comparing with a light wavelength energy of a mixed light or a short wavelength in the blue or green region. For Haematococcus, the producing concentration of astaxanthin, one of the cartenoids having a good antioxidative activity, may be changed according to the light wavelength region used for the culturing.

In addition, when the microalgae are exposed to ultraviolet rays for a long time, the growing property is known to be hindered.

Accordingly, in order to increase the growing property of the microalgae and remove unnecessary wavelength for the growing, exposure to only a specific wavelength region may be efficient.

For example, the productivity of expensive cartenoids such as β-carotene, lutein, astaxanthin from the microalgae may be increased by supplying a specific wavelength region.

A photobioreactor including an outer wall of a plurality of layers having different light filtering properties may be provided as a ninth embodiment of the present invention. The plurality of the layers may have different transmitting wavelength regions from each other.

Referring to FIG. 19A and 19B, the photobioreactor in accordance with the ninth embodiment of the present invention may be formed to include the first layer 2 and the second layer 3 of the culturing vessel 10-9 to transmit light wavelength in different wavelength regions. In the eighth embodiment, the transmitting amount of the light may be controlled regardless of the specific wavelength. In the ninth embodiment, the transmission of the specific wavelength region may be blocked to illustrate a selective transmitting property. Meanwhile, in order to prevent the forming of water drops, water repellent coating may be conducted at the total or a portion of the upper portion of the culturing vessel.

When the culturing vessel 10-9 of the photobioreactor in accordance with the present invention is floated on the sea and the first layer 2 having the light filtering function is exposed to the light source, for example, the sun, a portion of the wavelength region may be selectively penetrated or blocked from the supplied sunlight to the first layer 2. Accordingly, a light of a specific wavelength region may be concentrically supplied to the microalgae accommodated in the reaction chamber 1.

When a light of another wavelength region is required to be supplied to the microalgae, the culturing vessel 10-9 may be overturned to change the positions of the layers. Then, the second layer 3 may become the upper layer and may be exposed to the light source to achieve the requirement.

In this case, the ‘wavelength region’ may be for example, classified into a blue color line, a red color line, or a green color line, etc among the sunlight wavelength.

The wavelength region for transmitting or blocking may be appropriately selected according to the kind of the microalgae.

The layer having the light filtering function may be formed by mixing a plastic or a polymer material with a chemical component absorbing a light of a specific wavelength region. The chemical component may be included in a coloring pigment and available chemical components according to the coloring pigments are illustrated in Table 1.

TABLE 1

| pigments | Chemical components according to colors |
|---|---|
| Yellow pigments | lead chromate (PbCrO₄), yellow iron oxide |
| | (FeO(OH) or Fe₂O₃H₂O), cadmium yellow (CdS or |
| | CdS + ZnS), titanium yellow (TiO₂NiOSb₂O₃) |
| Orange pigments | chrome orange (PbCrO₄PbO), molybdenum orange |
| | (PbCrO₄PbMoO₄PbSO₄) |
| Red pigments | red iron oxide (Fe₂O₃), red lead oxide |
| | (Pb₃O₄), cadmium red (CdS + CdSe) |
| Violet pigment | manganese violet (NH₄MnP₂O₇) |
| Blue pigments | Prussian blue (Fe(NH₄)Fe(CN)₆xH₂O), |
| | ultramarine blue (Na_{6~8}Al₆Si₆O₂₄S_{2~4}), cobalt |
| | blue (CoOAl₂O₃) |
| Green pigments | chrome green (lead chromate + Prussian |
| | blue), emerald green (Cu(CH₃CO₂)₂Cu(AsO₂)₂) |

Natural coloring pigments may include gardenia yellow, polygonum indigo blue, Broussonetia kazinokii blue, carthamus red, Schisandra chinesis red, gallnut yellow, mugwort green, astaxanthin, anthocyanin, picoerythrin, xanthophyl, fucoxanthin, phycocyanin resveratrol, carotenoid, benzoquinone, shikonin, alazanine, anthraquinone, naphtoquinone, flavin, isoflavin and a mixture of two or more of them.

Selectively, the first layer 2 may be obtained by coating a painting composition including the pigments illustrated in Table 1 on a light transmitting layer, or by attaching a light filtering film or a tape. The light filtering film or the tape may be an optical film or a tape designed for transmitting or blocking a specific wavelength region.

In exemplary embodiments, light filtering patterns having various shapes as illustrated in FIGS. 20A to 20C may be formed on the light transmitting layer to obtain a plurality of layers having different light filtering properties.

The culturing vessel 10-10 of the photobioreactor in accordance with the tenth embodiment of the present invention may include a plurality of layers having different reflectivity from each other.

In exemplary embodiments, as illustrated in FIG. 22, a first layer 2 is positioned at an upper part and a second layer 3 is positioned at a lower part. The second layer 3 may reflect again the light transmitted through the first layer 2.

The light wavelength penetrating the first layer 2 and reaching the second layer 3 may not be supplied to the microalgae. However, the second layer 3 may reflect the light wavelength to the reaction chamber 1 again to supply the light to the microalgae. Accordingly, the supplying efficiency of the finally supplied light to the microalgae 7 with respect to the penetrating light through the first layer 2 may be increased.

The second layer 3 may be formed by laminating a reflecting member 8 having a high reflectivity on a portion of the light transparent layer. In this case, the reflecting member may be a reflecting film or a reflecting tape including one side having a high reflectivity.

The reflecting member may irregularly reflect the light to uniformly spread the light wavelength into an inner space of the reaction chamber 1. At the surface of the reflecting member 8, an embossing for improving the irregular reflection may be additionally formed.

Meanwhile, a portion of or a whole of the culturing vessel may be additionally coated with water repellent to prevent the generation of humidity or a water drop.

The culturing vessel of the photobioreactor in accordance with eleventh embodiment of the present invention may include an outer wall including a plurality of layers selectively having different penetrating materials.

Particularly, a first layer 2 of the culturing vessel 10-9 contacting an atmosphere in FIGS. 19A and 19B, may be formed by using a semi-permeable layer possibly penetrating external atmosphere, oxygen, carbon dioxide, etc and a second layer 3 contacting or submerged into the seawater or the fresh water may be formed by using a semi-permeable layer possibly penetrating water, nutrient salts, etc., however, blocking the microalgae.

In this case, when the microalgae accommodated in the culturing vessel 10-9 formed by using the semi-permeable layers is floating on the seawater or the fresh water, materials necessary for the culturing may be naturally supplied to the microalgae from an external environment even though isolated from the external environment.

Particularly, carbon dioxide in the atmosphere may be introduced into the reaction chamber 1 through the first layer 2 provided at the upper surface portion from the surface of the water while floating. The carbon dioxide may be consumed during the photosynthesis by the microalgae accommodated in the reaction chamber 1. Oxygen generated from the photosynthesis may be exhausted out to the atmosphere through the first layer 2.

Meanwhile, the inflow and outflow of the nutrient salts from the seawater or the fresh water may be possible through the second layer 3 contacting the seawater or the fresh water. In addition, the excreta exhausted from the microalgae while growing and the metabolite inhibiting the growth of the microalgae may dissolve into the seawater or the fresh water and may be naturally discharged along with the seawater or the fresh water. Accordingly, a separate purification work or a medium exchanging work is not necessary.

The outer wall of the photobioreactor in accordance with exemplary embodiments may be formed by using a transmittive material and may include a glass, a plastic or a polymer material, a semi-permeable layer, etc.

The photobioreactor may be formed in various shapes and may be selected from the group consisting of a rectangular, a flat plate, a cylinder reactor. Any shape may be possible in a wide enlargeable space like the ocean without limitation only when the photobioreactor has an appropriate shape for applying the natural energy, the sunlight for the growth of the microalgae.

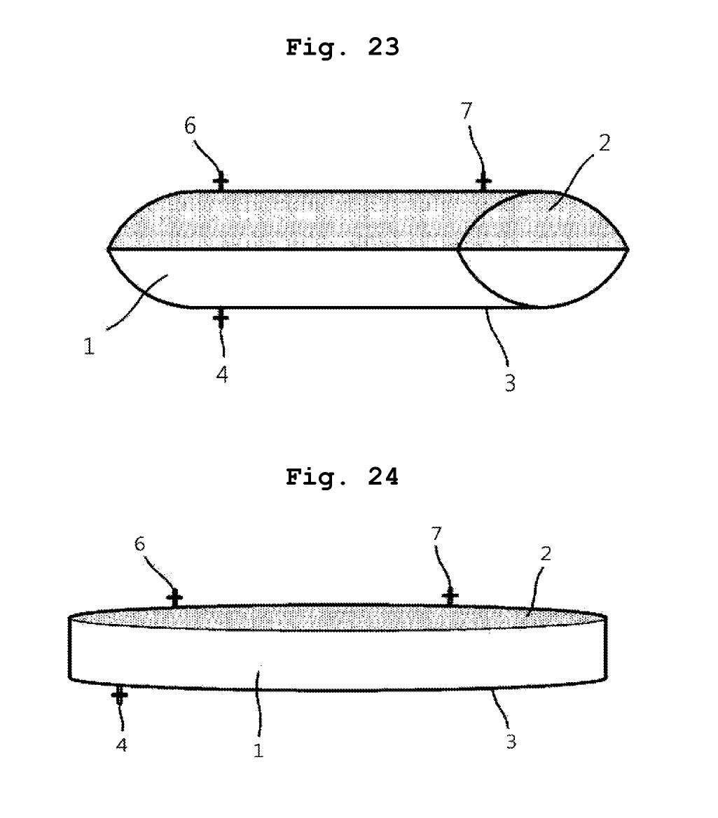

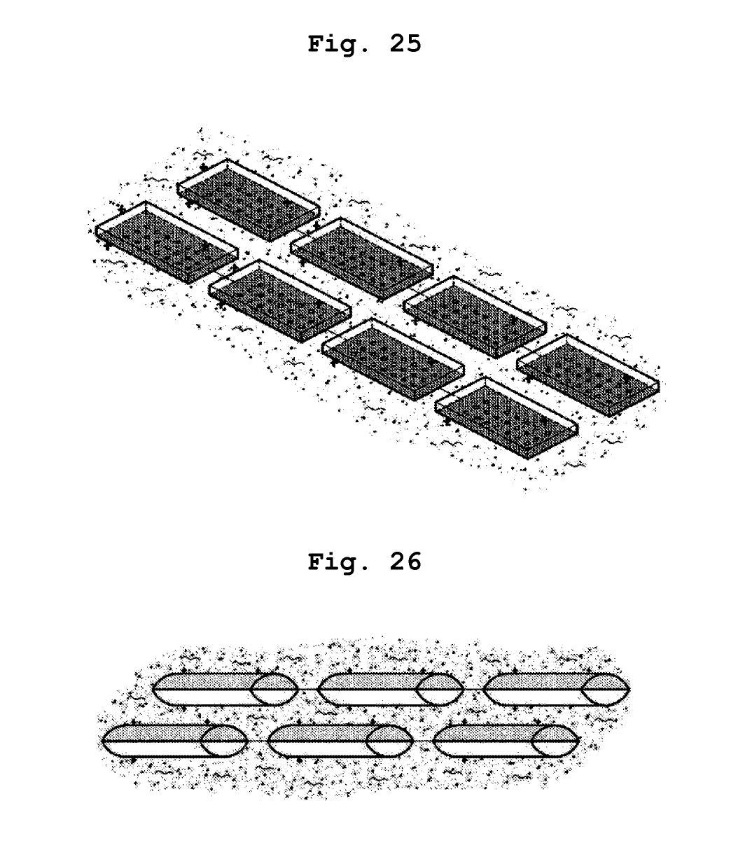

FIGS. 23 and 24 are exemplary diagrams on culturing vessels of a photobioreactor having a cylinder shape and an ellipse shape in accordance with an embodiment of the present invention.

Meanwhile, the photobioreactor may be floating itself, however, may additionally include a floating unit for floating the reactor and a fixing member for fixing the reactor within a designated position.

Selectively, in accordance with another exemplary embodiment, the culturing vessel may be connected to a structure on the water, a underwater structure or the surface under the water (not illustrated) through a combining unit (not illustrated) instead of a floating unit 30. The combining unit may include a rope, an iron chain, a steel wire, etc. The structure on the water may include an aquafarm equipment, a buoy, a light buoy, an underwater plant, a floating wave absorbing revetment, a barge, or a mega-float. The underwater plant may include a wind power generation equipment, a tide power generation equipment, a wave power generation equipment, a marine heliport or an oil prospecting ship. The underwater structure may include a cable, a gas pipe or an oil pipeline.

The above described exemplary embodiments utilize the sunlight, however, an artificial light source, for example, a light emitting diode may be used without restriction according to the same manner.

The photobioreactor for the mass culturing of the microalgae, having a filtering function to a designated wavelength region in exemplary embodiments may be applicable for the mass production of the microalgae as the following process.

For the mass culturing, a plurality of the above described photobioreactors may be installed as a group.

FIGS. 25 and 26 illustrate a group of plate type photobioreactors and a group of cylindrical type photobioreactors.

The foregoing is illustrative of example embodiments and is not to be construed as limiting thereof. Although a few example embodiments have been described, those ordinary skilled in the art will readily appreciate that many modifications are possible in the example embodiments without materially departing from the novel teachings and advantages of the invention. Accordingly, all such modifications are intended to be included within the scope of the invention as defined in the claims.

Explanations of letters or numerals
10, 10-1 to 10-11: culturing vessels

| | | | |
|---|---|---|---|
| 1: | reaction chamber | 2: | first layer |
| 3: | second layer | 4: | gas inlet |
| 5: | gas outlet | 6: | sampling port |
| 7: | microalgae | 8: | reflecting member |
| 6: | sampling port | 11: | outer wall |
| 13: | light filtering region, | | |
| | heat transforming region | | |
| 13a: | light filtering pattern, | | |
| | heat transforming pattern | | |
| 17: | cylindrical portion | 19: | conical portion |
| 20: | shape keeping frame | 25: | rope |
| 30, 30′: | floating unit | 35: | floating member |
| 37: | connecting frame | 39: | connecting ring |
| 40: | gas supplying pipe | 50: | gas supplying unit |
| 60: | weighing pendulum | 70: | fixing unit |

### INDUSTRIAL APPLICABILITY

The photobioreactor in accordance with the present invention is appropriate for a commercial mass culturing of microalgae for producing bio energy. While conducting the culturing, a quantitative amount of carbon dioxide which is the main culprit of an environmental problem concerning the global warming may be consumed. Accordingly, the culturing is environment-friendly.

In addition, instead of using a light of a designated wavelength from an artificial light source, the selection of a specific wavelength region may be easily accomplished by blocking or transmitting a specific wavelength region of the limitless natural light source, the sunlight, through painting, coating, mixing with a coloring material, etc. a culturing vessel material. A remarkable increase of the concentration of the metabolite produced from the microalgae may be accomplished.

1. A photobioreactor for mass culturing of microalgae, comprising:
a culturing vessel comprising an outer wall including a light filtering region for selectively transmitting or blocking a portion of wavelength or region of sunlight and/or a heat transforming region for transforming selectively absorbing a portion of the wavelength into heat, and a reaction chamber as a three dimensional inner space defined by the outer wall for accommodating the microalgae; and
a floating unit for positioning the culturing vessel at just below a surface of water or at an appropriate depth in the water, or a combining unit for connecting the culturing vessel with a surface under the water, an underwater structure, or a structure on the water, in order to expose the culturing vessel to an appropriate intensity of the sunlight for growing the microalgae and in order to protect from waves.

2. The photobioreactor of claim 1, wherein a whole or a portion of the outer wall includes a transparent material.

3. The photobioreactor of claim 2, wherein the transparent material is one of glass, polyvinyl chloride (PVC), polyethyleneterephthalate (PET), acryl, polystyrene (PS), high density polyethylene (HDPE), low density polyethylene (LDPE), polypropylene (PP), polycarbonate (PC), polyamide (PA), or a laminate structure of two or more of them..

4. The photobioreactor of claim 1, wherein the whole or the portion of the outer wall includes a semi-permeable material.

5. The photobioreactor of claim 4, wherein the semi-permeable material is at least one polymer selected from the group consisting of cellulose acetate, cellulose triacetate, cellulose acetate-cellulose triacetate blends, nitrocellulose, gelatin, polyamine, polyimide, poly(ether imide), aromatic polyamide, polybenzimidazole, polybenzimidazolone, polyacrylonitrile, polyacrylonitrile-poly(vinyl chloride) copolymer, polysulfone, polyethersulfone, poly(dimethylphenylene oxide), poly(vinylidene fluoride), polyelectrolyte complexes, polyolefin, poly(methyl methacrylate), polyvinyl alcohol, and a copolymer thereof

6. A photobioreactor for mass culturing of microalgae, comprising:
a culturing vessel comprising an outer wall including a light filter region for selectively transmitting or blocking a portion of wavelength or region of sunlight and/or a heat transforming region for transforming selectively absorbing a portion of the wavelength into heat, and a reaction chamber as a three dimensional inner space defined by the outer wall for accommodating the microalgae, the outer wall including a first layer and a second layer apart from the first layer and having a different property from the first layer; and
a floating unit for positioning the culturing vessel at just below a surface of water or at an appropriate position in the water, or a combining unit for connecting the culturing vessel with a surface under the water, an underwater structure, or a structure on the water, in order to expose the culturing vessel to an intensity of the sunlight appropriate for growing the microalgae and in order to protect from waves.

7. The photobioreactor of claim 6, wherein light transmission properties of the first layer and the second layer are different from each other.

8. The photobioreactor of claim 6, wherein wavelength regions of a light penetrating the first layer and the second layer are different from each other.

9. The photobioreactor of claim 6, wherein the first layer and the second layer have different material penetrating properties from each other.

10. The photobioreactor of claim 9, wherein the first layer includes a semi-permeable layer which selectively penetrates oxygen or carbon dioxide, and the second layer includes a semi-permeable layer which selectively penentrates water and nutrient salts.

11. The photobioreactor of claim 6, wherein the first layer and the second layer have different light reflecting properties from each other and wherein the light wavelength penetrated through the first layer is reflected by the second layer so as to supply into the reaction chamber.

12. The photobioreactor of claim 1, wherein the light filtering region or the heat transforming region is a pattern having a predetermined shape.

13. The photobioreactor of claim 12, wherein the pattern is designed to change the shape or density in accordance with an input of light energy.

14. The photobioreactor of claim 12, wherein the light filtering region or the heat transforming region comprises:
a light transparent layer; and
a light blocking pattern layer or a heat transforming pattern layer attached on one side of the outer wall.

15. The photobioreactor of claim 14, wherein the light blocking pattern layer or the heat transforming pattern layer is a tape possibly attached or detached from the light transparent layer.

16. The photobioreactor of claim 1, wherein the light filtering region is obtained by painting, coating or mixing a coloring agent.

17. The photobioreactor of claim 16, wherein the light filtering region is obtained by mixing one pigment selected from the group consisting of lead chromate (PbCrO₄), yellow iron oxide (FeO(OH) or Fe₂O₃H₂O), cadmium yellow (CdS or CdS+ZnS), titanium yellow (TiO₂NiOSb₂O₃), chrome orange (PbCrO₄PbO), molybdenum orange (PbCrO₄PbMoO₄PbSO₄), red iron oxide (Fe₂O₃), red lead oxide (Pb₃O₄), cadmium red (CdS+CdSe), manganese violet (NH₄MnP₂O₇), Prussian blue (Fe(NH₄)Fe(CN)₆xH₂O), ultramarine blue (Na_{6·8}Al₆Si₆O₂₄S_{2˜4}), cobalt blue (CoOAl₂O₃), chrome green (lead chromate+Prussian blue), emerald green (Cu(CH₃CO₂)₂Cu(AsO₂)₂), gardenia yellow, polygonum indigo blue, Broussonetia kazinokii blue, carthamus red, Schisandra chinesis red, gallnut yellow, mugwort green, astaxanthin, anthocyanin, picoerythrin, xanthophyl, fucoxanthin, phycocyanin resveratrol, carotenoid, benzoquinone, shikonin, alazanine, anthraquinone, naphtoquinone, flavin, isoflavin and a mixture of two or more of them.

18. The photobioreactor of claim 1, wherein the heat transforming region includes a light heat transforming material.

19. The photobioreactor of claim 18, wherein the light heat transforming material absorbs infrared rays, near infrared rays, visible rays or ultraviolet rays.

20. The photobioreactor of claim 18, wherein the light heat transforming material is an organic or inorganic pigment or dye, an organic coloring agent, a metal, a metal oxide, a metal carbonate or a metal borate.

21. The photobioreactor of claim 1, wherein a plurality of the light filtering region is arranged horizontally or vertically one by one, so as to transmit two or more wavelength regions within the sunlight region so as to possibly control a wavelength ratio.

22. The photobioreactor of claim 1, wherein the light filtering region is arranged in a lattice pattern, so as to transmit two or more wavelength regions within the sunlight region so as to possibly control a wavelength ratio.

23. The photobioreactor of claim 1, wherein the structure on the water includes an aquafarm equipment, a buoy, a light buoy, an underwater plant, a floating wave absorbing revetment, a barge, or a mega-float.

24. The photobioreactor of claim 1, further comprising a shape keeping frame, disposed at an inside or outside of the culturing vessel to keep a three dimensional shape of the culturing vessel.

25. The photobioreactor of claim 1, wherein the culturing vessel and the floating unit are connected by using at least one length adjustable rope, and wherein a weighing pendulum clings to a bottom portion of the culturing vessel, s pendulum weight and a rope length being controlled to control a depth of the culturing vessel into the water.

26. The photobioreactor of claim 1, further comprising a gas supplying pipe connected to the bottom portion of the culturing vessel and a gas supplying unit for supplying external air through the gas supplying pipe to the culturing vessel, the microalgae being uniformly dispersed within the culturing vessel through a mixing reaction of bubbles.

27. The photobioreactor of claim 1, wherein the culturing vessel has an inverse conical shape and the gas supplying pipe and the weighing pendulum are connected to an apex of the conical culturing vessel.

28. The photobioreactor of claim 26, wherein the culturing vessel includes an upper vertical cylindrical portion and a lower inverse conical portion, and the gas supplying pipe and the weighing pendulum are connected to an apex of the inverse conical portion.

29. The photobioreactor of claim 25, wherein at least two of the culturing vessels are arranged above and below and the weighing pendulum is cling to at least one of the culturing vessels including a lowermost culturing vessel.

30. The photobioreactor of claim 1, wherein the floating unit includes at least one pair of connecting points, the culturing vessel has a length larger than a width, the connecting points where both end portions in lengthwise direction make a pair, are connected by at least one rope so that the culturing vessel is overturned freely by waves or movements of seawater.

31. The photobioreactor of claim 1, wherein the culturing vessel is formed to have an elongated cylindrical shape in parallel to a surface of the water, and is connected to the floating unit, the underwater structure, the structure on the water or the surface under the water.

32. The photobioreactor of claim 1, wherein the culturing vessel is formed to have a middle cylindrical portion and a pair of conical portions at both ends of the middle cylindrical portion, a rope being connected to each of apexes of the conical portions.

33. A method of culturing microalgae comprising:
(a) injecting culture medium in a culturing vessel of one of photobioreactors of claim 1, and inoculating microalgae;
(b) sealing the culturing vessel, fixing the culturing vessel to a floating unit and then putting the culturing vessel into seawater or fresh water; and
(c) irradiating light to the microalgae for photosynthesis.

34. The photobioreactor of claim 1, wherein a portion of or a whole of the culturing vessel is coated with water repellent.

35. The photobioreactor of claim 1, wherein the underwater structure is a cable, a gas pipe or an oil pipeline.
